(19) United States
(12) Patent Application Publication
(10) Pub. No.: US 2016/0089228 A1
(43) Pub. Date: Mar. 31, 2016

(54) TEMPORARY EMBOLIC PROTECTION DEVICE AND METHODS THEREOF

(71) Applicant: CONTEGO MEDICAL, LLC, Raleigh, NC (US)

(72) Inventors: Ravish Sachar, Raleigh, NC (US); Udayan Patel, San Jose, CA (US)

(21) Appl. No.: 14/865,796

(22) Filed: Sep. 25, 2015

Related U.S. Application Data
(60) Provisional application No. 62/055,226, filed on Sep. 25, 2014.

(51) Int. Cl. A61F 2/01; A61B 17/221; A61M 25/10

(52) U.S. Cl. CPC A61F 2/013; A61M 25/104; A61B 17/221; A61F 2002/016; A61F 2002/011; A61B 2017/22001

(57) ABSTRACT

A percutaneous transluminal temporary embolic protection device includes an embolic filter mounted to a guidewire shaft at a location proximate the distal end of the guidewire. The filter can be positioned down-stream from a thrombectomy treatment site at a target location and can be properly positioned to capture embolic particles that may be set loose into the blood stream as the thrombectomy procedure is performed. The embolic filter is normally undeployed against the guidewire shaft to facilitate introduction and withdrawal of the device to and from the target location

### CROSS-REFERENCE TO RELATED APPLICATIONS

The present disclosure claims priority to U.S. Patent Application No. 62/005,226, filed Sep. 25, 2014, entitled “Temporary Embolic Protection Device and Methods Thereof,” which is incorporated herein by reference in its entirety.

### BACKGROUND

1. Field of the Invention

Implementations described herein relate generally to surgical devices and relate more specifically to temporary embolic protection devices and associated methods.

2. Related Art

Deep vein thrombosis (DVT) can be described as the formation of a blood clot or thrombus in a deep vein, predominately in the legs. Similarly, subclavian vein and axillary vein thrombosis (ASVT) are described as the formation of a blood clot or thrombosis in the subclavian vein or axillary veins between the clavicle and ribs. Pulmonary embolism is caused by the detachment or embolism of a clot that travels to the lungs. Together, DVT, ASVT and pulmonary embolism constitute a single disease process known as venous thromboembolism. Thrombectomy is a procedure used to break up clots and can be a percutaneous, catheter-based procedure. Percutaneous thrombectomy devices can be categorized as rotational, rheolytic or ultrasound enhanced. No matter the operational modality, distal embolism is a risk inherent to thrombectomy. Accordingly, a need exists for improved temporary embolic protection devices and associated methods.

### SUMMARY

It is to be understood that this summary is not an extensive overview of the disclosure. This summary is exemplary and not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

Stated generally, the present disclosure comprises a percutaneous transluminal temporary embolic filter and is intended for use as an adjunct to medical procedures where distal embolization is a risk.

Stated more specifically, the present disclosure comprises a catheter having an elongated shaft, proximal and distal ends, a longitudinal axis and a filter. The filter comprises a first ring coaxially fixedly mounted on a distal portion of the catheter shaft, a second ring coaxially slidably mounted on a distal portion of the catheter shaft and configured to be moved toward and away from the first ring and a scaffolding extending between the first and second rings. The scaffolding further comprises a plurality of first longitudinal connecting members, each having a first end attached to the first ring and a second end extending toward the second ring; a plurality of second longitudinal connecting members, each having a first end attached to the second ring and a second end extending toward the first ring. The filter further comprises a membrane connected to at least the scaffolding.

In an additional aspect, the present disclosure is directed to a wire-in-wire configuration including a guidewire and an embolic filter coupled to the guidewire. The embolic filter can include a first ring located proximate the distal end of the guidewire and a second ring located between the distal end of the guidewire and a proximal end of the guide wire. The filter can further include a filter membrane coupled to the first and second rings, where the filter membrane can be movable between an undeployed configuration and a deployed configuration upon displacement of the first and second rings relative to each other. The filter can also include an actuator wire extending through a central channel provided in the guidewire where the actuator wire is coupled to one of the first ring or the second ring such that activation of the wire results in a corresponding displacement of the first or second ring. The filter can further include a filter chasis or scaffolding comprising at least one strut extending between and coupled to the first ring and the second ring such that the strut bows outward from the guidewire, deploying the filter membrane, as a distance between the first and second ring decreases.

In another aspect, the temporary embolic filter can comprise a wire-in-wire configuration comprising an outer wire having a lumen with an inner wire movably disposed therein. In this aspect, the filter is constructed substantially identically to the filter described above except that the distal-most first collar is located on a portion of the inner wire extending past the distal terminal end of the outer wire and the second collar is located on a distal portion of the outer wire. In operation, causing the inner wire to move proximally relative to the outer wire causes the filter to move from an undeployed configuration to a deployed configuration and vice-versa.

Additional features and advantages of exemplary implementations of the disclosure will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

The accompanying drawings, which are incorporated in and constitute a part of this specification, illustrate aspects and together with the description, serve to explain the principles of the methods and systems.

FIG. 1 illustrates a side view of one aspect of a temporary embolic filter.

FIG. 2A illustrates a cross-section of the proximal end of the device with integral embolic filter shown in FIG. 1; and FIG. 2B illustrates a cross-section of the distal end of the device shown in FIG. 1.

FIG. 3A illustrates a schematic view of one aspect of a filter scaffolding of the embolic filter device of FIG. 1, showing the filter scaffolding in an un-deployed position.

FIG. 3B illustrates a schematic view of the filter scaffolding of FIG. 3A, showing the filter scaffolding in a deployed position.

FIG. 4A illustrates a schematic view of one aspect of a filter scaffolding of the embolic filter device of FIG. 1, showing the filter scaffolding in an un-deployed position.

FIG. 4B illustrates a schematic view of the filter scaffolding of FIG. 4A, showing the filter scaffolding in a deployed position.

FIG. 5 illustrates a schematic view of another aspect of a filter scaffolding of the embolic filter device of FIG. 1, showing the filter scaffolding in an un-deployed position.

FIG. 6 illustrates a schematic view of the filter scaffolding of FIG. 5, showing the filter scaffolding in a deployed position.

FIG. 7 illustrates a schematic view of a third aspect of a filter scaffolding of the embolic filter device of FIG. 1, showing the filter scaffolding in an un-deployed position.

FIG. 8 illustrates a schematic view of the filter scaffolding of FIG. 7, showing the filter scaffolding in a deployed position.

FIG. 9 illustrates another embodiment of a temporary vascular filter having a wire-on-wire configuration.

FIG. 10A illustrates another example of an embolic filter including an outer sleeve.

FIG. 10B illustrates another view of the embolic filter of FIG. 4A.

FIG. 11 illustrates a blood vessel having a stenosis.

FIG. 12 illustrates the blood vessel with stenosis of FIG. 11 with the embolic filter device of FIG. 1 positioned therein.

FIG. 13 illustrates the blood vessel and embolic filter device of FIG. 11 with the integral embolic filter expanded.

FIG. 14 illustrates the blood vessel and embolic filter device of FIG. 11 with the integral embolic filter deployed.

FIG. 15 illustrates the blood vessel and device of FIG. 11 after treatment of the stenosis, with the embolic filter still in its deployed position.

FIG. 16 illustrates the blood vessel and device of FIG. 11 after treatment of the stenosis, with the embolic filter in an un-deployed position in preparation for withdrawal of the device from the vessel.

FIG. 17 illustrates an alternate aspect of a filter scaffolding comprising a sinusoidal frame.

FIG. 18 illustrates one aspect of the attachment of the sinusoidal frame.

### DETAILED DESCRIPTION

The present invention can be understood more readily by reference to the following detailed description, examples, drawing, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention provided as an enabling teaching of the invention in its best, currently known aspect. To this end, those skilled in the relevant art will recognize and appreciate that many changes can be made to the various aspects of the invention described herein, while still obtaining the beneficial results described herein. It will also be apparent that some of the desired benefits described herein can be obtained by selecting some of the features described herein without utilizing other features. Accordingly, those who work in the art will recognize that many modifications and adaptations to the present invention are possible and can even be desirable in certain circumstances and are a part described herein. Thus, the following description is provided as illustrative of the principles described herein and not in limitation thereof.

Reference will be made to the drawings to describe various aspects of one or more implementations of the invention. It is to be understood that the drawings are diagrammatic and schematic representations of one or more implementations, and are not limiting of the present disclosure. Moreover, while various drawings are provided at a scale that is considered functional for one or more implementations, the drawings are not necessarily drawn to scale for all contemplated implementations. The drawings thus represent an exemplary scale, but no inference should be drawn from the drawings as to any required scale.

In the following description, numerous specific details are set forth in order to provide a thorough understanding described herein. It will be obvious, however, to one skilled in the art that the present disclosure may be practiced without these specific details. In other instances, well-known aspects of percutaneous transluminal devices and embolic filters have not been described in particular detail in order to avoid unnecessarily obscuring aspects of the disclosed implementations.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

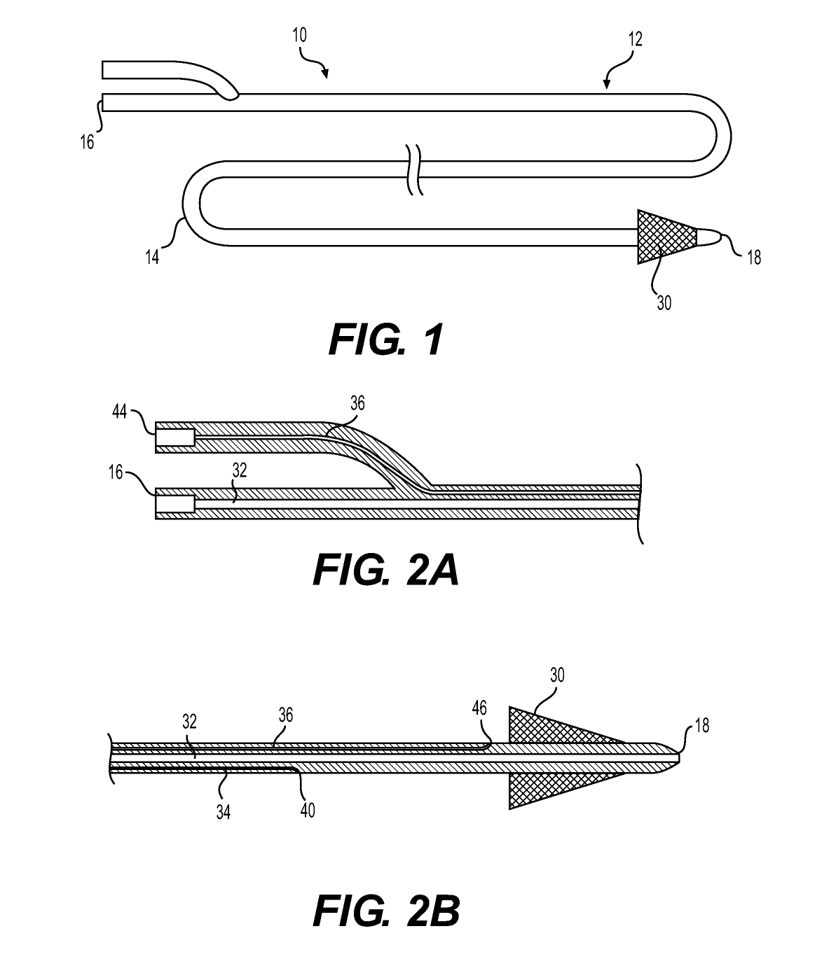

Referring now to the drawings, in which identical numbers indicate identical elements throughout the various views, FIG. 1 illustrates a first aspect of a catheter with integral embolic filter 10 according to the present invention. The catheter with integral embolic filter 10 comprises an elongated catheter 12 having a shaft 14 with a proximal end 16 and a distal end 18. As used herein, “proximal” refers to the portion of the device closest to the physician performing the procedure and “distal” refers to the portion of the device that is furthest from the physician performing the procedure. The shaft 14 of the catheter 12 is sized and configured to slidably receive a thrombectomy treatment device (e.g., an angioplasty balloon, a mechanical thrombosis device, an ablation device, or any other tool or surgical device known in the art for treatment of thrombosis). As illustrated in the figures, and in contrast to previous treatment catheter, the shaft 14 of the catheter 12 does not include any integrated treatment features, e.g., an angioplasty balloon coupled to/extending from the shaft of the catheter to treat the thrombosis. Instead, the shaft 14 of the present catheter 12 is sized to accommodate a treatment device slidable along the shaft 14. Thus allowing the catheter 12 to guide the treatment device to the target location. The use of a slidable and detachable treatment device also allows for a greater variety of thrombectomy treatment devices as the system is no longer limited to the catheter's treatment modality (e.g., catheter with an integral angioplasty balloon where the balloon and/or inflation port of the balloon could block access/movement of the device along the catheter shaft).

An embolic filter 30 can be mounted to the catheter shaft 14 at or proximal to the distal end 18 of the catheter 12. In additional or alternative embodiments, the filter 30 can be oriented to face towards or away from the treatment device. One skilled in the art will also appreciate in light of the present disclosure that the catheter can be configured to be, for example and without limitation, an over-the-wire catheter, a rapid-exchange catheter and the like. It is solely for clarity of disclosure that the present description describes an over-the-wire catheter modality.

Referring now to FIG. 2, the catheter shaft 14 can define two lumens: a main lumen 32 and an embolic filter actuator wire lumen 36. The main lumen 32 can extend from the proximal end 16 to the distal end 18 of the catheter shaft 14. The main lumen 32 can optionally provide a working channel and be configured to receive a guidewire therethrough for advancing the distal end 18 of the catheter 12 through the patient's vasculature to a target location. As used herein, the term “target location” refers to a location downstream to the occlusion within the patient's vasculature being treated. The actuator wire lumen 36 can extend from a proximal port 44 at the proximal end 16 of the catheter 12 and through the catheter shaft 14 to a distal port 46.

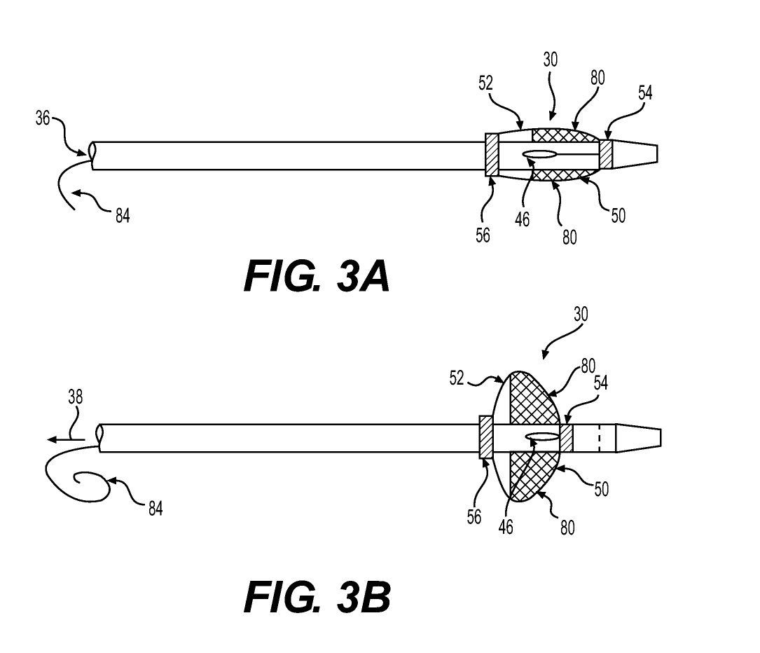

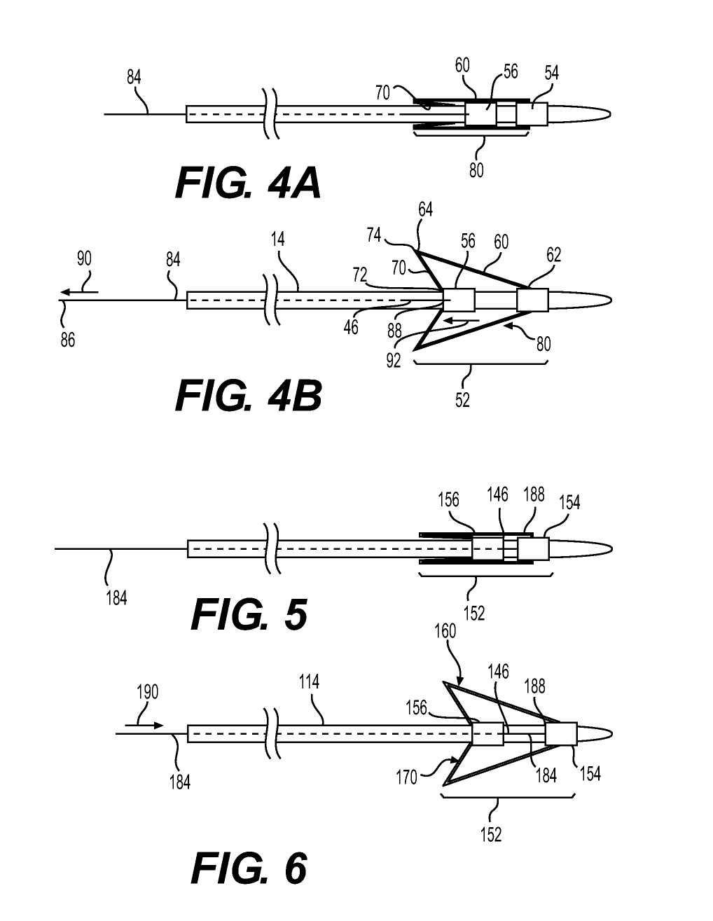

Referring to aspects of the present disclosure illustrated in at least FIGS. 3A through 4B, the embolic filter 30 comprises a filter membrane 50 having holes selectively sized to permit the passage of blood but to capture particles larger than normal blood particles and a filter chassis or scaffolding 52, for supporting the filter membrane. For clarity of illustration, many of the drawing figures omit the filter membrane 50 when illustrating the filter chassis or scaffolding, but it will be understood that all embolic filters disclosed in this application comprise a filter membrane supported by the filter chassis or scaffolding. It is contemplated that the chassis or scaffolding 52 can include a proximal ring 56 and a distal ring 54. In operation, movement of the proximal ring 56 toward and away from the distal ring 54 to open and to close the embolic filter 30 can be accomplished by manipulation of an actuator wire 84. In one aspect, the proximal end 86 of the actuator wire 84 can extend out of the proximal port 44 of the actuator wire lumen 36 so as to be controllable by the physician performing the procedure. Here, the actuator wire 84 can extend through the actuator wire lumen 36 and can exit through the distal port 46 of the actuator wire lumen. It is contemplated that the distal end 88 of the actuator wire 84 can be attached to at least one of the distal ring 54 or the proximal ring 56.

In one aspect, the distal ring 54 can be fixed in place on the catheter shaft 14, and the proximal ring 56 can be slidably mounted to the catheter shaft for axial movement in the proximal and distal directions. In another aspect, the proximal ring 56 can be fixed in place on the catheter shaft 14, and the distal ring 54 can be slidably mounted to the catheter shaft for axial movement in the proximal and distal directions.

In one aspect illustrated in FIGS. 3A and 3B, the filter chassis 52 comprises a plurality of ribs or struts 80 spaced circumferentially about and connected to the proximal and distal rings, 56 and 54, respectively, each strut having a first end and a second end. The first end of each strut 80 can be attached to the distal ring 54 and the second end of each strut can be attached to the proximal ring 56. In operation, when the relative distance between the distal ring and proximal ring decreases, the struts 80 will bow outward, erecting the filter membrane 50 as shown in FIG. 3B. In one exemplary aspect, the distal port 46 of the actuator wire lumen 36 is located between the proximal ring 56 and the distal ring 54. Here, the proximal ring is fixed relative to the catheter and the distal ring, which is operably coupled to the actuator wire, is movable along the axis of the catheter. In operation, the actuator wire would be pulled proximally 58 to move the distal ring towards the proximal ring, causing the struts to bow outward and move the filter from an undeployed position to a deployed position.

In another aspect illustrated in FIGS. 4A and 4B, the filter chassis or membrane can comprise a plurality of struts 80 that further comprise a plurality of first strut sections 60 and a plurality of second strut sections 70. Each of the plurality of first strut sections 60 can have a first end 62 and a second end 64. The first end 62 of each first strut section 60 can be attached to the distal ring 54, and each first strut section can extend in the proximal direction. Each of a corresponding plurality of second strut sections 70 can have a first end 72 and a second end 74. Here, the first end 72 of each second strut section 70 can be attached to the proximal ring 56, and each second strut section can also extend in the proximal direction. The second end 64 of each first strut section 60 can attach to the second end 74 of a corresponding second strut section 70. As one skilled in the art will appreciate, a plurality of strut 80 can be spaced circumferentially about and connecting the proximal and distal rings to form the scaffolding 52. In operation and as shown in FIGS. 4A and 4B, when the proximal and distal rings 56, 54 are adjacent one another each strut 80 can be configured to fold back upon itself. Additionally, when the proximal ring 56 is proximally displaced from the distal ring 54, the struts 80 can be configured to open in a manner similar to an umbrella. The filter membrane 50 can be supported on the first strut sections 60 such that when the scaffolding 52 opens, as shown in FIG. 4B, the filter membrane can deploy in a manner similar to an umbrella canopy. In contrast to the embolic filter 30 depicted in FIG. 3B, the deployed filter depicted in FIG. 4B (and FIGS. 6, 8 and 10) defines an arrow-shaped profile. That is, the filter membrane 50, extends along both the first and second strut sections 60, 70 at an acute angle with respect to the shaft 14. This structure prevents material captured by the embolic filter 30 from being released when the embolic filter 30 is removed from the patient. For example, particles can be trapped by the filter membrane 50 extending along the first strut 60, second strut 70 and/or the shaft 14 of the catheter.

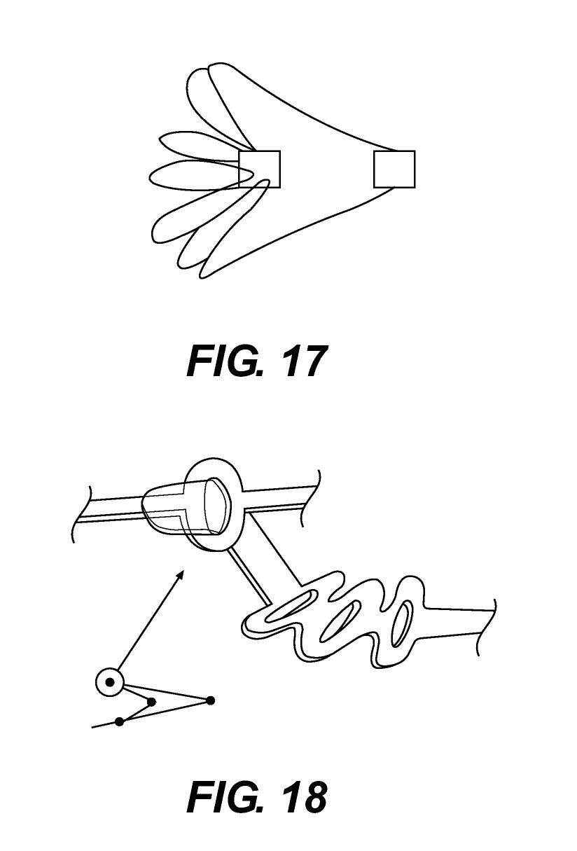

In yet other aspects, the plurality of second strut sections 70 can be replaced with a sinusoidal ring structure 55 as illustrated in FIGS. 17-18. In this aspect, the sinusoidal ring 55 contracts radially inward as the relative distance between the distal and proximal rings increases and expands as the relative distance between the distal and proximal rings decreases.

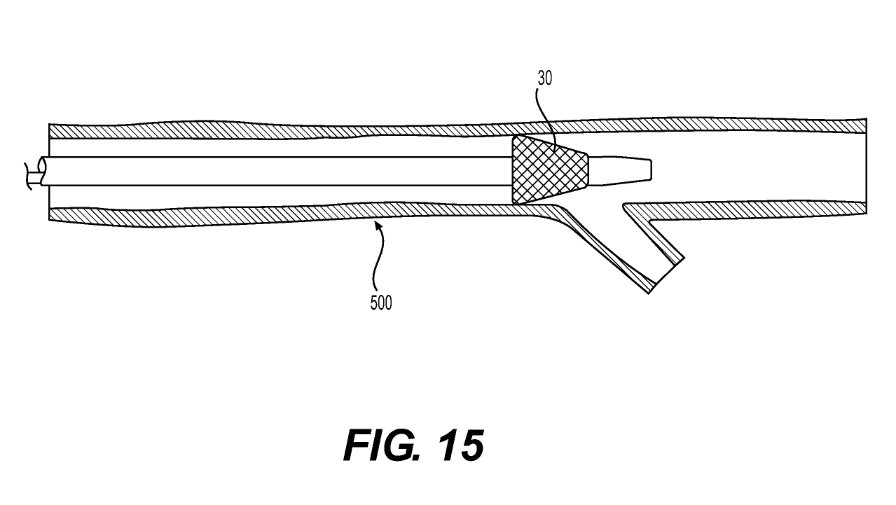

It is contemplated that each strut can further comprise at least one “zone of weakness,” i.e., a zone of the strut that can be configured to be physically weaker than the majority of the strut in order to control the locations at which the struts bend. One skilled in the art will appreciate that the at least one zone of weakness can be formed in any of a number of ways. In one aspect, a notch can be formed in one or both sides of the strut. In another aspect, at least one of the upper surface and lower surface of the strut can be scored. In another aspect, the at least one zone of weakness can be formed of a material that can be structurally weaker than the material comprising the remainder of the strut. In yet other aspects, the at least one zone of weakness can comprise mechanical hinges. In yet other aspects and as shown in FIG. 15, the apices of the sinusoidal ring 55 comprise a zone of weakness. In even further aspects, at least two of these approaches can be combined to form the at least one zone of weakness, e.g., both notching the width and scoring the depth of the strut. In addition, the at least one zone of weakness can comprise a plurality of one type of physical arrangement, e.g., a single zone of weakness can comprise a plurality of notches or a plurality of scores. In operation, the at least one zone of weakness can be configured to bend the strut in response to a force at a predetermined angle to the longitudinal axis of that portion of the strut.

One skilled in the art will appreciate here are a variety of ways in which the filter scaffolding 52 and actuator wire 84 can be arranged to permit the embolic filter 30 to be opened and closed by moving the proximal end 86 of the actuator wire. In a first aspect, the filter scaffolding 52 can be formed in a normally closed or undeployed position. In operation, pulling the proximal end 86 of the actuator wire 84 can cause the proximal ring 56 to slide in a proximal direction to open the filter scaffolding 52. The filter scaffolding can be configured so that releasing the tension on the actuator wire 84 and/or pushing the actuator wire 84 distally can permit the filter scaffolding 52 to collapse to an un-deployed position.

In another aspect of the present disclosure illustrated in FIGS. 5 and 6, a filter scaffolding 152 can comprise a proximal ring 156 that can be fixed with respect to a catheter shaft 114 and a distal ring 154 that can be slidably positioned along the catheter shaft in the proximal and distal directions. In a further aspect, a distal port 146 of an actuator wire lumen 136 can be located distal to the proximal ring 156. Here, an actuator wire (not shown) can extend through the actuator wire lumen, can exit through a distal port 146, and can attach to the distal ring 154. The filter scaffolding 152 can be formed in a normally closed position. In operation, pushing the actuator wire 184 can displace the distal ring 154 in a distal direction away from the proximal ring 156 to deploy the filter scaffolding 152. The filter scaffolding can be configured so that releasing the force on the actuator wire 184 and/or pushing the actuator wire 184 distally can permit the filter scaffolding 152 to return to its un-deployed position.

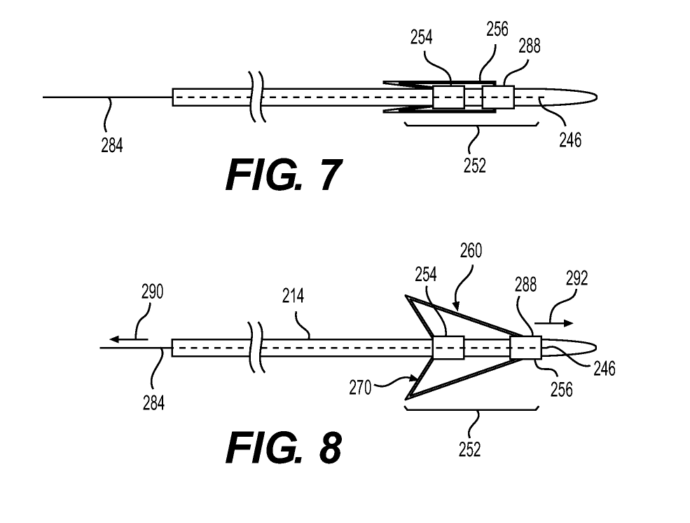

In yet another aspect of the present disclosure illustrated in FIGS. 7 and 8, a proximal ring 254 can be fixed with respect to a catheter shaft 214, and a distal ring 256 can be slidably positioned along the catheter shaft in the proximal and distal directions. In a further aspect, a distal port 246 of an actuator wire lumen 236 can be located distal to the distal ring 256. Here, an actuator wire 284 can extend through the actuator wire lumen 236, can exit through the distal port 246, and can attach to the distal ring 256. As illustrated in FIGS. 4-8, the distal port 46, 146, 246 can be located offset from the distal end of the catheter shaft 14, 114, 214. Accordingly, the actuator wire lumen 36, 146, 246 can terminate before the distal end of the catheter shaft 14, 114, 216 thereby providing a solid nose portion extending distally from the termination of the actuator wire lumen 36 and/or the distal port 46, 146, 246.

The filter scaffolding 252 can be formed in a normally closed position. In operation, pulling on the actuator wire 284 can displace the distal ring 256 in a distal direction and away from the proximal ring 156 to deploy the filter scaffolding 252. The filter scaffolding can be configured so that releasing the force on the actuator wire 284 can permit the filter scaffolding 252 to return to its un-deployed position.

Referring back to FIGS. 4A and 4B, another aspect of a filter scaffolding can be structurally identical to the first embodiment 52 except that the filter scaffolding can be formed in a normally open or deployed position. Here, it is contemplated that application of a distally directed force to the proximal end 86 of the actuator wire 84 (i.e., pushing the actuator wire) can maintain the proximal ring 56 in its distal position and hence can maintain the filter scaffolding 52 in its un-deployed position. The filter scaffolding 52 can be permitted to expand to its normally deployed position, expanding the filter membrane 50, upon release of the force applied to the actuator wire 84. Immediately after completion of the interventional procedure, a distally directed force can again be applied to the proximal end 86 of the actuator wire 84, moving the proximal ring 56 toward the distal ring 54 and collapsing the filter scaffolding 52.

Referring back to FIGS. 5 and 6, a fifth aspect can be structurally identical to the third aspect with the exception that the filter scaffolding 152 can be formed in a normally open position. Here, it is contemplated that the distal ring 154 can be normally displaced toward the distal end 18 of the catheter shaft 114. In operation, pulling on the distal end 188 of the actuator wire 184 can move the distal ring 154 proximally toward the fixed proximal ring 156, collapsing the filter scaffolding 152 while releasing the tension on the actuator wire 184 can permit the filter scaffolding 152 to expand to its deployed position.

While FIGS. 1-8 are described above as illustrating a catheter including an integral embolic filter 10, it is contemplated that the catheter could be replaced by a guidewire such that the apparatus would comprise a wire-in-wire configuration, i.e., a guidewire including an integral embolic filter, and an actuator wire extending within a central lumen of the guidewire. Accordingly, without repeating the description of each of FIGS. 1-8 as provided above, it is contemplated that each of those figures are also illustrative of a guidewire including an integral embolic filter 10. Each of the additional elements described above are considered the same. For example, the system illustrated in FIGS. 3A and 3B can include an embolic filter 30 comprising a filter membrane 50 having holes selectively sized to permit the passage of blood but to capture particles larger than normal blood particles and a filter chassis or scaffolding 52, for supporting the filter membrane. The chassis/scaffolding 52 can include a proximal ring 56 and a distal ring 54. In operation, movement of the proximal ring 56 toward and away from the distal ring 54 to open and to close the embolic filter 30 can be accomplished by manipulation of an actuator wire 84. In one aspect, the proximal end 86 of the actuator wire 84 can extend out of the proximal port 44 of the actuator wire lumen 36 provided on the guidewire, such that the actuator wire 36 is controllable by the physician performing the procedure. The actuator wire 84 can extend through the actuator wire lumen 36 and can exit through the distal port 46 of the actuator wire lumen. The distal end 88 of the actuator wire 84 can be attached to at least one of the distal ring 54 or the proximal ring 56. The distal ring 54 can be fixed in place on the catheter shaft 14, and the proximal ring 56 can be slidably mounted to the catheter shaft for axial movement in the proximal and distal directions. Alternatively, the proximal ring 56 can be fixed in place on the catheter shaft 14, and the distal ring 54 can be slidably mounted to the catheter shaft for axial movement in the proximal and distal directions.

In general, a guidewire is constructed from a smaller (diameter) and more rigid material than a catheter. Similar to catheters, guidewires provide torque control, flexibility and the ability to support the passage of another device or system over it. Due to their structure, guidewires generally provide better trackability (ability navigate vasculature) and steerability.

As will be described below, because a guidewire has a smaller outer diameter than a catheter, a greater variety of thrombectomy tools and treatment devices and be provided over the guidewire to access the treatment position. The tool/treatment device may be movable over the guidewire in multiple directions over guidewire, i.e., axially along the guidewire toward/away from the distal end of the guidewire, rotationally around the diameter of the guidewire. The thrombectomy tool/treatment device can include an angioplasty balloon, a mechanical thrombosis device, an ablation device, or any other tool or surgical device known in the art for treatment of thrombosis.

The increased sized (diameter) of the catheter can increase the possibility of ostial trauma and vascular complications. For vascular treatment, catheter diameters generally range from 4 F to 25 F (outer diameter ranging from 0.055 inches to 0.345 inches), selection depending various factors including the age of the patient and the size of the vessels. In contrast, for vascular treatment, guidewire diameters generally range from 0.010 inches to 0.060 inches. Typically, a physician will choose the smallest diameter catheter feasible to minimize the risk of trauma or complications during the procedure. In contrast, because guidewires have a much smaller diameter than catheters, the diameter of the guidewire is a less significant factor in selection. Instead, selection is guided by vessel anatomy, devices to be used/passed over the guidewire, and physician preference. In the present system, it is contemplated that the guidewire can have an outer diameter between 0.010 inches to 0.060 inches. In another example, the outer diameter can vary between 0.012 inches and 0.045 inches. In yet another example, the outer diameter can vary between 0.014 inches and 0.035 inches.

A catheter is generally described as a hollow flexible tube that is inserted into the body, duct or vessel over a guidewire. The flexibility of a catheter typically necessitates the use of a guidewire. In the present example, because the embolic filter is integral with the guidewire, the system does not require an additional guidewire or other guiding device to direct movement and location of the filter. The flexibility/stiffness of a catheter or a guidewire defines the characteristics of the wire a measure of its elastic modulus and can be measured in terms of its flexural modulus. Flexibility/stiffness varies, for example, in relation to the material properties, core diameter, and physical structure of the catheter/guidewire. The stiffness of a catheter used in vascular treatment ranges from 3.0 g to 50.0 g. In contrast, stiffness of a guidewire used in vascular treatment can range from 1.5 g to 14.0 g. For example, polymer-covered (hydrophilic) wires such as an Abbott HT Pilot® 50 guidewire can have a stiffness of 1.5 g. An Abbott HT Pilot® 150 and 200 can have a stiffness of 2.7 g and 4.1 g, respectively. An Abbott HT Progress® 40, 80, 120 can have a stiffness of 4.8 g, 9.7 g, 13.9 g, respectively. A Boston Scientific Choice PT® can have a stiffness of 1.9 g. Non-covered (non-lubricious) coil guide wires, such as Abbott HT Cross-IT® 100XT can have a stiffness of 1.7 g. An Abbott Miraclebros® can have various stiffness, including, 3.9 g, 4.4 g, 8.8 g, and 13.0 g. A Confianza Pro® can have a stiffness of 9.3 g and 12.4 g. And a Medtronic Persuader® 3, 6 can have a stiffness of 5.1 and 8.0, respectively. Similarly, the flexural modulus of a guidewire used in vascular treatment can range from 9.5 Gpa to 158.4 Gpa. For example, a plain Amplatz type wire has a stiffness of 9.5 GPa. A “heavy duty” Amplantz type wire has a stiffness ranging from 11.4 GPA to 14.5 GPa. A “stiff” Amplantz type wire has a stiffness of 17 GPa. An “extra stiff” Amplantz type wire has a stiffness of 29.2 GPa. A “super stiff” Amplantz type wire has a stiffness of 60.3 GPa. An “ultra stiff” Amplantz type wire has a stiffness of 65.4 GPa. A Backup Meier® wire has a stiffness of 139.6 GPa. A Lunderquist® “extra stiff” wire has a stiffness of 158.4 GPa.

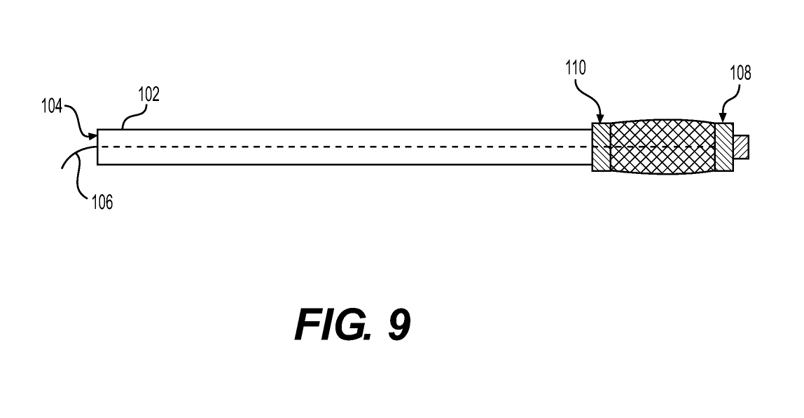

In another aspect illustrated in FIG. 9, the temporary embolic filter 100 can comprise a wire-in-wire configuration comprising an outer wire 102 having a lumen 104 with an inner wire 106 movably disposed therein. In this aspect, the filter 106 is constructed substantially identically to the filter described above except that the distal-most first collar 108 is located on a portion of the inner wire extending past the distal terminal end of the outer wire and the second collar 110 is located on a distal portion of the outer wire. In operation, causing the outer wire to move proximally relative to the inner wire causes the filter to move from an undeployed configuration to a deployed configuration and vice-versa.

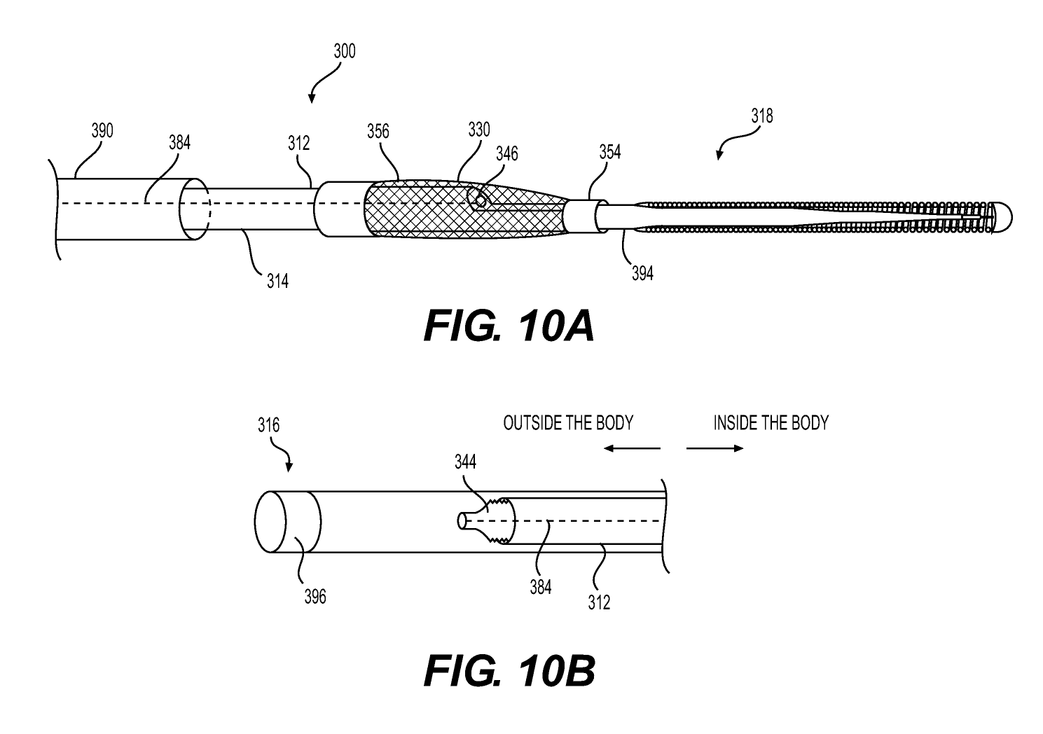

In another aspect it may be desirable for the embolic filter and corresponding catheter/guidewire to remain in the patient for an extended period of time, e.g., more than just temporary placement/treatment. Accordingly a catheter/guidewire may be provided with an outer sleeve that permits longer term placement within the patient. FIGS. 10A and 10B illustrate a guidewire with integral embolic filter 300 including an outer sleeve 390. The outer sleeve 390 extends over the guidewire 312 and provides a protective barrier between the guidewire 312 and the patient.

The guidewire with integral embolic filter 300 can include similar components and structure to those described above with respect to the catheters/guidewires illustrated in FIGS. 1-9. For example, similar to the catheters/guidewires depicted in in FIGS. 1-9, guidewire with integral embolic filter 300 provided in FIGS. 10A and 10B can comprise an elongated guidewire 312 having a shaft 314 with a proximal end 316 and a distal end 318. The embolic filter 330 can be mounted on the guidewire shaft 314 at proximate the distal end 318 of the guidewire 312. Because the embolic filter 330 is coupled directly to the guidewire 312, the guidewire 312 can be advanced through the patient's vasculature to a target location without the assistance of an additional locating guidewire. The guidewire 312 can be composed of a highly trackable and steerable material such as nitinol.

As outlined above, the embolic filter 330 comprises a filter membrane 350 and a filter chassis or scaffolding 352, for supporting the membrane. The chassis/scaffolding 352 can include a proximal ring 356 and a distal ring 354. In operation, movement of the proximal ring 356 toward and away from the distal ring 354 cause the embolic filter 330 to open and close. Either the distal ring 354 or the proximal ring 356 can be fixed to the guidewire shaft 314, with the other ring sildably mounted to the guidewire shaft 314 for axial movement in the proximal and distal directions. As provided above, the chassis/scaffolding 352 can include a plurality of rips or struts (and/or a plurality of strut sections) spaced circumferentially around the guidewire 312 and coupled to the proximal and distal rings 356,354. Each strut can further comprise a “zone of weakness” to control the locations at which the struts bend. The plurality of strut section can also be replaced with a sinusoidal ring structure as illustrated in FIGS. 17-18.

In operation, movement of the proximal ring 56/distal ring 354 toward and away from each other can be accomplished by manipulation of an actuator wire 384. The guidewire 312 can include an actuator wire lumen 336 that extends from the proximal end 316 to a location proximate the distal end 318 of the guidewire 312. The actuator wire lumen 336 can extend from a proximal port 344 at the proximal end 316 of the guidewire 312, through the guidewire shaft 314, to a distal port 346. The actuator wire 384 can be accessed at the distal port 346 such that the wire can be moved in the proximal and distal directions. As illustrated in FIG. 10B, the actuator wire 384 can be coupled to an actuator screw 392 located near the proximal port 344. Rotation of the actuator screw 392 can result in corresponding axial movement of the actuator wire 384 in the proximal and distal directions. It is also contemplated that the actuator wire 384 can be manipulated without the use of an actuator screw 392. For example, access to the wire is provided at the distal port 346 where the actuator wire 384 is manipulated either directly or via the use of a tool. As illustrated in FIG. 10B, the outer sleeve can be include an end cap 396 for sealing the opening provided at the end of the outer sleeve 309. If an actuator screw 392 is not utilized, the actuator wire 384 can be fixed to the end cap such that its proximal/distal location is fixed with the end cap is in closed position.

As illustrated in FIG. 10A, the distal port 346 is located between the proximal ring 356 and the distal ring 354. Here, the proximal ring 356 is fixed relative to the guidewire 312 and the distal ring 354. The actuator wire 384, extending through the actuator wire lumen 336 is operably coupled to the distal ring 354, which is movable along the axis of the guidewire 312. In operation, the actuator wire 384 is pulled proximately to move the distal ring 354 towards the proximal ring 356, causing the embolic filter 330 to bow outward and move from an undeployed position to a deployed position. In another example, not illustrated, the actuator wire 384 is operably coupled to the proximal ring 356, which is movable along the axis of the guidewire 312. In operation, the actuator wire 384 is pulled proximately to move the proximate ring 356 towards the distal ring 354, causing the embolic filter 330 to bow outward and move from an undeployed position to a deployed position.

As illustrated in FIG. 10A, the distal port 346 is located proximate the distal end 318 of the guidewire 312. It is further contemplated that the distal port 346 for the actuator wire 384 can be located at any position along the guidewire 312. For example, the distal port 346 can be located at the extreme distal end of the guidewire 312 or at a position between the proximal end 316 and the proximal ring 356. As provided in FIG. 10A, the guidewire 312 includes a reduced diameter portion 394 adjacent the distal end 318. The actuator wire lumen 336 can extend through the reduced diameter portion 394 or, as illustrated in FIG. 10A, the actuator wire lumen 336 can extend only through the increased diameter portion with the distal port 346 located on a surface of the increased diameter portion of the guidewire 312. The reduced diameter portion 394 can provide a solid nose portion of the guidewire 312 to assist in navigating the guidewire 312 to the target location.

As illustrated in FIGS. 10A and 10B, the guidewire with integral embolic filter 300 includes an outer sleeve 390. The outer sleeve 390 extends over the guidewire 312 and provides a protective barrier between the guidewire 312 and the patient. Use of the outer sleeve 390 permits long-term/indefinite placement of the filter 300 within the patient. The outer sleeve 390 can be configured to permit relative movement between the outer sleeve 390 and the guidewire 312 such that the expanded filter 330 can remain stationary within the patient, despite movement of the patient and/or outer sleeve 390 with respect to the guidewire 312. For example, outer sleeve 390 can be constructed from a semi-rigid material and low friction material including, for example, a plastic or metal material such as stainless steel, nitinol, polyolefins, polyesters, polyurethanes, florinated polymers, or any other material known in the art, The outer sleeve 390 can be constructed from a low friction material and/or include a coating that permits relative movement between the outer sleeve 390, the guidewire 312 and the patient. For example, the outer sleeve 390 can have a polytetrafluoroethylene (PTFE), polyethylene furanoate (PEF), or hydrophilic coating. The outer sleeve 390 can also be sized to permit relative movement between the outer sleeve 390, the guidewire 312, and the patient. For example, the outer sleeve 390 can have an inner diameter greater than the outer diameter of the guidewire 312. In one example, the outer sleeve 390 can have an outer diameter of 0.035 inches, an inner diameter of 0.029 inches, with a resulting wall thickness of 0.003 inches. The guidewire 312 can have an outer diameter of 0.027 inches, providing a 0.001 clearance around the perimeter of the guidewire 312. The example guidewire 312 can also have an inner diameter of 0.013 inches, with a resulting wall thickness of 0.007 inches.

As outlined above, it is contemplated that various thrombectomy tools/treatment devices can be movable (in multiple directions) over the guidewire 312. Likewise, because the combined outer sleeve 390 and guidewire 312 has an outer diameter smaller than a catheter, it is contemplated that various thrombectomy tools/treatment devices can be provided over the combined sleeve 390/guidewire 312.

In yet another aspect, the temporary embolic filter can have a braided nitinol scaffold and, in a further aspect, the scaffold can be configured with a baseline memory in the undeployed configuration. The scaffold can be coupled to a membrane comprising a finely-brained nitinol wire and, in a further aspect, the membrane can be coupled to the inner surface of the scaffold. In a further aspect, the membrane can have a baseline memory in the deployed configuration. In operation, when the scaffold is activated and deployed by the operator, the filter membrane will urge towards its baseline, deployed configuration but will be controllably constrained by the scaffold.

In those aspects in which the force applied to the actuator wire is configured to be an axial compressive force, those skilled in the art can appreciate that a stiffer wire can be used to prevent buckling of the actuator wire than in those embodiments where the force applied to the actuator wire is configured to be an axial tensile force.

In the present disclosure, and especially in the case of actuator wires, the term “wire” is intended to comprise, for example and without limitation, metallic wires, polymeric wires, and the like. In the case of polymeric wires, the polymers used can comprise, for example and without limitation, nylon, polypropylene and the like.

In the foregoing aspects, the filter chassis or scaffold can be formed from any material known to be suitable, including shape-memory materials such as, for example and without limitation, nitinol. It is also contemplated that the scaffold components can be laser cut, formed from braided elements or any other method known in the art.

In the foregoing aspects, the filter membrane 50 can be formed from at least one of a textile, a polymer and a wire mesh or braid. In one non-limiting aspect, the filter membrane can be formed from braided nitinol wire and, in a further aspect, can have a baseline shape corresponding to either a deployed or undeployed configuration. In another aspect, the filter membrane 50 comprises pores and, in a further aspect, the pores can be sized to allow blood to pass but not embolic particles. It is also contemplated that the filter membrane 50 can be mounted either on top of or inside of the frame. It is contemplated that the filter membrane 50 and chassis/scaffolding can have a deployed diameter up to 50 mm or approximately 2 inches.

In the foregoing aspects, the filter membrane 50 can be configured to cover the exterior surface of the outermost strut sections, i.e., the first strut sections 60, 160, and 260. Optionally, the filter membrane 50 can be further configured to extend beyond the distal or second ends 64, 164, and 264, 364 of the first strut sections 60, 160, and 260, where it can be attached to the circumference of the distal ring 54, 154, 254. In those aspects in which the distal ring can be fixed, the filter membrane 50 can optionally be configured to extend beyond the distal end of the distal ring and can be attached to the circumference of the catheter/guidewire shaft 14, 114, 314 at a location between the distal ring 54, 154, 254 and the distal end of the catheter/guidewire shaft.

It is also contemplated that the filter membrane 50 in each of the disclosed embodiments can be attached to the inner surfaces of the first strut sections 60, 160, and 260 instead of to the outer surfaces.

It is further contemplated that the inner or second strut sections 70, 170, 270 can also be configured in a concave shape with respect to the blood flow when the filter scaffolding is deployed. In further or additional aspects, the filter membrane 50 can be attached to the inner or outer surfaces of the second strut sections 70, 170, 270. When the filter membrane 50 is attached to the surfaces of the second strut sections 70, 170, 270, the filter membrane 50 can optionally extend beyond the distal or second ends 74, 174, 274 of the second strut sections and be attached to the circumference of the proximal ring 56, 156, 256, 356. It is also contemplated that, if the filter membrane 50 can be attached to the outer surfaces of the second strut sections 70 and the proximal ring 56, 156, 256, 356 can be fixed, the filter membrane can be configured to extend beyond the distal end of the proximal ring and can be attached to the catheter shaft 14 at a location between the proximal and distal rings 56, 54.

In all of the foregoing instances, the filter scaffolding comprises a fixed ring and a movable ring, raising the filter can be accomplished by moving the rings apart, and collapsing the filter can be achieved by moving the rings together or vice-versa. “Moving apart” and “moving together” are used as relative terms, such that only one of the two rings need move with respect to the other ring for the rings to “move apart” or “move together.”

Similarly, the process of raising and collapsing the filter can be thought of as being viewed from the perspective of the catheter, such that a movable ring can be moved toward or away from a fixed ring.

In all of the foregoing instances, one can appreciate that both actively applying a force to move a ring and releasing a force to permit the ring to move of its own accord comprise a step of “causing” the movable ring to move by “controlling” the actuator wire. Thus, in both the normally deployed and normally un-deployed filter scaffolding embodiments described herein, the actuator wire can be “controlled” to “cause” a movable ring to move, whether that control takes the form of applying or releasing a force on the actuator wire.

It is also contemplated that, rather than having the physician directly grasp the proximal end of the actuator wire, a control device can be associated with the proximal end of the actuator wire at the proximal end of the catheter shaft. The control device can incorporate, for example and without limitation, levers, sliders, rotating spindles, or the like to facilitate movement of the wire. One example of such a mechanical arrangement is described in U.S. Patent Publication No. US 2010/0106182, paragraphs [0079]-[0090] and FIGS. 29-33, which disclosure is hereby incorporated by reference.

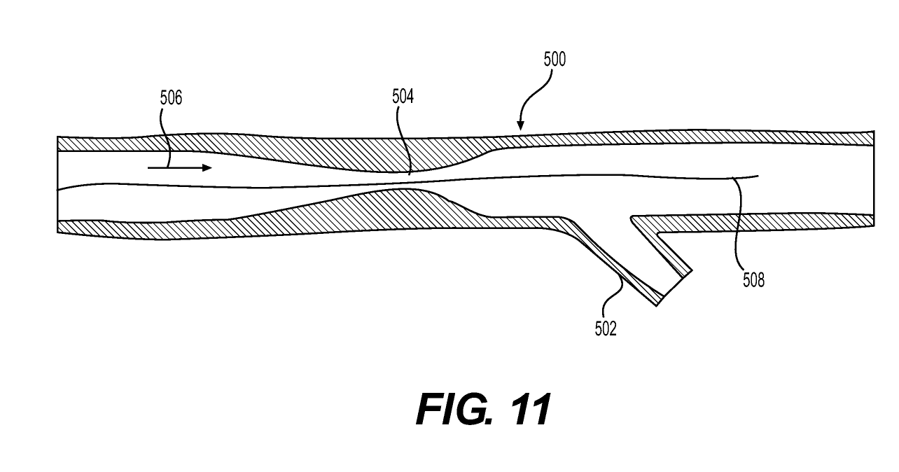

Use of the temporary embolic filter described above to prevent an embolism in a blood vessel can be shown in FIGS. 11-15. In FIG. 11, a vessel 500 can have a branch vessel 502 diverging from it. The vessel 500 can have a stenosis 504. The direction of blood flow through the vessel 500 is indicated by the arrow 506. A guide wire 508 can be inserted by the physician as a preliminary step in the interventional procedure when using a catheter with integral embolic filter (as noted above, an introductory guidewire is not necessary when a guidewire with integral embolic filter is used).

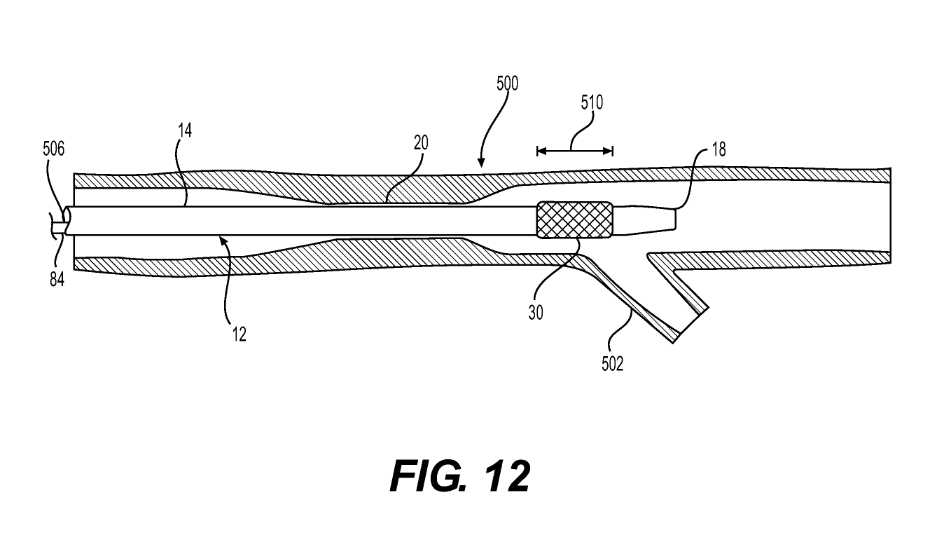

FIG. 12 shows the catheter/guidewire 12 with embolic filter 30 in its un-deployed position and lying adjacent to the catheter/guidewire shaft 14. The distal end 18 of the catheter 14 has been advanced over the guide wire 506 until the un-deployed embolic filter is at the target location. Similarly, the distal end 18 of the guidewire shaft 14 can be advanced through the vessel 500 until the un-deployed embolic filter is at the target location.

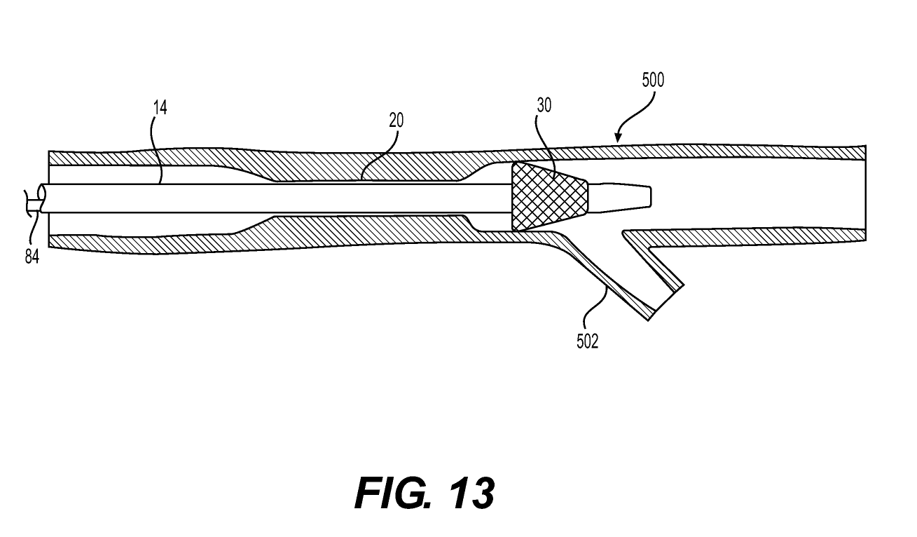

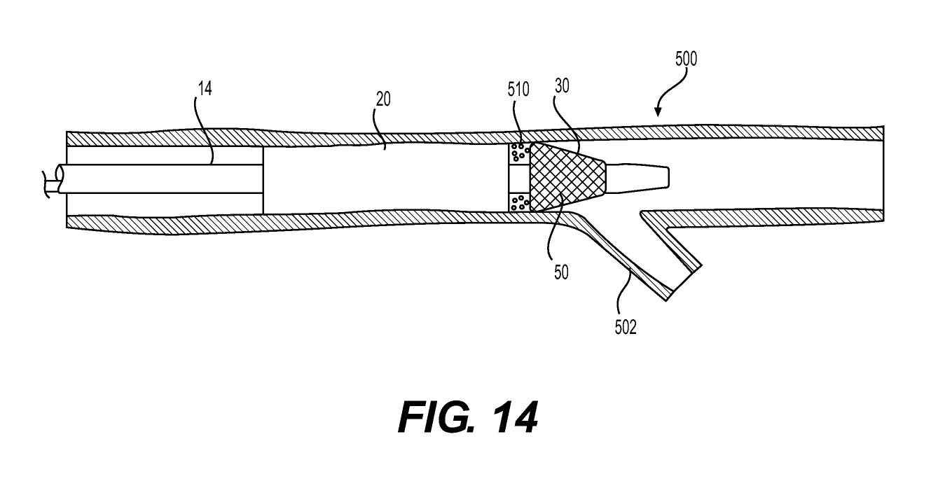

In FIG. 13 the embolic filter 30 has been expanded by pulling on the actuator wire 84. In FIG. 14, a thrombectomy device 20 is deployed. (For the sake of clarity of the present invention, the thrombectomy device is only abstractly represented.) In the process of thrombectomy, embolic particles 510 are released and swept by the blood flow into the open proximal end of the embolic filter 30, where they are captured by the filter membrane 50.

In FIG. 15, the formerly stenosed region can be open, and the thrombectomy device is removed. The embolic filter 30 remains open to capture any emboli released as the thrombectomy device is removed.

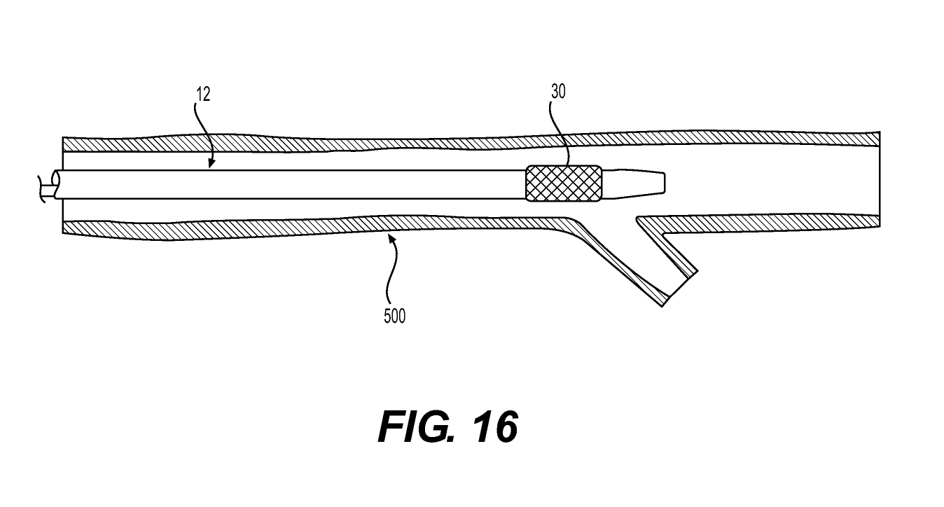

In FIG. 16, the embolic filter 30 can be closed, trapping captured emboli within the filter. The catheter 12 can now be withdrawn from the vessel 500.

One implementation of each of the disclosed embolic filters can be adjunct to treatment of an ilio-femoral DVT. Here, prior to insertion of the thrombectomy device, the temporary embolic filter would be inserted into and deployed in the inferior vena cava and used as described above. In another implementation, the disclosed embolic filters can be used in the subclavian vein and axillary vein while treating patients with arterio-venous (a-v) access thrombosis. In other implementations, it is contemplated that the disclosed embolic filters can be used in any vascular bed.

The present invention can thus be embodied in other specific forms without departing from its spirit or essential characteristics. The described aspects are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed:

1. An apparatus comprising:
an elongated guidable intravascular member; and
an embolic filter coupled to the member comprising:
a first ring located proximate a distal end of the member;
a second ring located between the distal end of the member and a proximal end of the member;
a filter membrane coupled to the first and second rings, the filter membrane movable between an undeployed configuration and a deployed configuration upon displacement of the first and second rings relative to each other;
an actuator wire extending through a central channel provided in the member and coupled to one of the first ring or the second ring such that activation of the wire results in a corresponding displacement of the first or second ring.

2. The apparatus of claim 1, wherein the member is a guidewire,
wherein the guidewire does not include any integrated treatment feature coupled to or extending from an outer surface of the guidewire,
wherein the outer surface of the guidewire is sized and configured to slidably receive a treatment device.

3. The apparatus of claim 2, wherein the guidewire has an outer diameter between 0.010 inches and 0.060 inches.

4. The apparatus if claim 2, wherein the actuator wire extends through an opening provided in the guidewire to engage at least one of the first ring or the second ring.

5. The apparatus if claim 2, wherein the first ring is fixedly coupled to the guidewire and the second ring is slidably coupled to the guide wire, wherein the actuator wire is coupled to the second ring.

6. The apparatus if claim 2, wherein the second ring is fixedly coupled to the guidewire and the first ring is slidably coupled to the guide wire, wherein the actuator wire is coupled to the first ring.

7. The apparatus if claim 2, wherein actuator wire is activated by movement of the wire in a direction towards the proximal end of the guide wire.

8. The apparatus if claim 2, wherein movement of the filter membrane to a deployed configuration causes filter membrane to extend in a direction away from the guidewire such that an outer diameter of the filter membrane is greater than an outer diameter of the guidewire.

9. The apparatus of claim 2, wherein the filter membrane defaults to an undeployed configuration.

10. The apparatus of claim 2, wherein the filter further includes at least one strut extending between and coupled to the first ring and the second ring, wherein the at least one strut bows outward from the guidewire, deploying the filter membrane, as a distance between the first and second ring decreases.

11. The apparatus of claim 2, wherein the filter further includes:
a strut extending between and coupled to the first ring and the second ring, the strut comprising:
a first strut section coupled to the first ring, and
a second strut section coupled to the second ring,
wherein the first strut section and the second strut section are hingedly connected at an end opposite an end coupled to the corresponding first and second ring,
wherein the strut bows outward from the guidewire, deploying the filter membrane, as a distance between the first and second ring decreases.

12. The apparatus of claim 11, wherein in the deployed configuration the second strut section extends at least partially under the first strut section such that at least a portion of the second strut section is located between the first strut section and the guidewire.

13. The apparatus of claim 1, wherein the member is a catheter,
wherein the catheter does not include any treatment feature coupled to or extending from an outer surface of the catheter,
wherein the outer surface of the catheter is sized and configured to slidably receive a treatment device.

14. An apparatus comprising:
an elongated guidable intravascular member;
an embolic filter coupled to the member comprising:
a first ring located proximate a distal end of the member;
a second ring located between the distal end of the member and a proximal end of the guide wire;
a filter membrane coupled to the first and second rings, the filter membrane movable between an undeployed configuration and a deployed configuration upon displacement of the first and second rings relative to each other;
an actuator wire extending through a central channel provided in the member and coupled to one of the first ring or the second ring such that activation of the wire results in a corresponding displacement of the first or second ring; and
a sleeve slidable over the member and the embolic filter in the undeployed configuration, the sleeve sized and configured to permit relative movement between the sleeve and the member.

15. The apparatus of claim 14, wherein the member is a guidewire,
wherein an outer surface of the guidewire is sized and configured to slidably receive a treatment device.

16. The apparatus of claim 14, further including an end cap for coupling to the proximal end of the sleeve such that the sleeve can indefinitely be position over the member.

17. The apparatus if claim 14, wherein the second ring is fixedly coupled to the member and the first ring is slidably coupled to the member,
wherein the actuator wire is coupled to the first ring.

18. The apparatus of claim 14, wherein the actuator wire extends through an opening provided in the member to engage at least one of the first ring or the second ring.

19. The apparatus of claim 18, wherein the opening is provided on a surface extending transverse to a longitudinal axis of the member.

20. The apparatus of claim 18, wherein the member includes a solid nose portion extending distally from a portion of the member proximate the opening provided for the actuator wire.

21. The apparatus of claim 14, further including a treatment device movable over the member to a treatment position,
wherein the treatment device includes one of an angioplasty balloon and a mechanical thrombosis device.

22. The apparatus of claim 14, wherein the filter includes at least one strut extending between and coupled to the first ring and the second ring, wherein the at least one strut extends outward from the member, deploying the filter membrane, as a distance between the first and second ring decreases.
